(19) United States
(12) Patent Application Publication
(10) Pub. No.: US 2025/0099299 A1
(43) Pub. Date: Mar. 27, 2025

(54) ELECTRONIC SURGICAL EYE IMPLANT INCLUDING SMART HAPTICS

(71) Applicant: GLAIVE MEDICAL OPTICS INC, SHREVEPORT, LA (US)

(72) Inventors: Wayne H. Woods, JR., Carlisle, MA (US); Christopher Shelby, Shreveport, LA (US)

(21) Appl. No.: 18/373,669

(22) Filed: Sep. 27, 2023

(51) Int. Cl. A61F 9/08

(52) U.S. Cl. CPC A61F 9/08

(57) ABSTRACT

A device configured to be implanted in an eye includes: an imaging system that receives visible light incoming to the eye; a projection system that generates and projects an image onto a retina of the eye in which the device is implanted, the image being based on the received visible light; and one or more haptics that include one or more electronic components, wherein the one or more haptics are movable relative to a central portion of the device such that the device can be arranged in a collapsed configuration and an expanded configuration.

### BACKGROUND

The present invention relates generally to ocular implants and, more particularly, to electronic surgical eye implants including smart haptics that can be used in both therapeutic and diagnostic applications.

Being able to target/stimulate specific areas of the retina surface is desirable and difficult to achieve. Approaches to doing this have included chips that directly interface with the neurons in the retina surface. In this disclosure, devices and methods are described that are much less surgically invasive compared to such alternatives.

### SUMMARY

In an aspect of the invention, there is a device configured to be implanted in an eye, the device comprising: an imaging system that receives visible light incoming to the eye; a projection system that generates and projects an image onto a retina of the eye in which the device is implanted, the image being based on the received visible light; and one or more haptics that include one or more electronic components, wherein the one or more haptics are movable relative to a central portion of the device such that the device can be arranged in a collapsed configuration and an expanded configuration.

In embodiments, the one or more electronic components comprises an inductive charging coil.

In embodiments, the one or more electronic components comprises a wireless communication antenna.

In embodiments, the one or more electronic components comprises an inductive charging coil and a wireless communication antenna.

In embodiments, the one or more haptics are pivotally connected to the central portion.

In embodiments, the one or more haptics are flexibly connected to the central portion.

In embodiments, the one or more electronic components extend continuously through the one or more haptics and the central portion.

In embodiments, the imaging system and the projection system are in the central portion.

In embodiments, the device further comprises a power source and control circuitry.

In embodiments, the power source and/or the control circuitry are integrated with the imaging system and the projection system.

In embodiments, the power source and/or the control circuitry are separate from the imaging system and the projection system.

In embodiments, the projection system comprises optical source generation circuitry and an optical phased array that is configured to project the image onto a determined location of the retina.

In embodiments, the projection system comprises a light generation panel and a microlens array that is configured to project the image onto a determined location of the retina.

In embodiments, the one or more electronic components comprise flexible electrically conductive elements that maintain their electrical continuity when the device is in the collapsed configuration, when the device is in the expanded configuration, and when the device is moved between the collapsed configuration and the expanded configuration.

In an aspect of the invention, there is a method comprising implanting the device into the eye. In embodiments, the implanting comprises: arranging the device in the collapsed configuration; inserting the device into the eye while the device is in the collapsed configuration; and arranging the device in the expanded configuration when the device is in the eye.

In an aspect of the invention, there is a method of using the device, the method comprising: causing the device to project a diagnostic image on different locations of the retina of the eye; receiving patient feedback for each of the different locations; creating a mapping of the retina of the eye based on the feedback; and programming the mapping into the device.

In embodiments, the method further comprises optimizing the mapping using artificial intelligence. In embodiments, the mapping maps the retina into functional areas and non-functional areas. In embodiments, the device is configured to control the projection system based on the mapping to project a beam onto a functional area of the retina to reduce or eliminate a scotoma caused by a non-functional area of the retina.

In an embodiment, a device according to any of the aspects above comprises a body made of acrylic and/or silicone lens material.

In an embodiment, a device according to any of the aspects above comprises a single piece lens.

In an embodiment, a device according to any of the aspects above comprises a body having dimensions of 1 mm<=TH<=3 mm and 1 mm<=W<=10 mm.

In an embodiment, in a device according to any of the aspects above comprises, the projection system comprises an optical phased array (OPA) that comprises components of an on-chip optical phase array including but not limited to: one or more splitters, waveguides, phase shifters, amplifiers, and emitting elements. In embodiments, the OPA is configured to generate and steer an optical beam defining the image in a desired direction onto a desired location of the retina. In an embodiment, in a device according to any of the aspects above comprises, the projection system comprises a light generation panel and a microlens array that are configured to generate and steer an optical beam defining the image in a desired direction onto a desired location of the retina.

In an embodiment, a device according to any of the aspects above comprises an imaging chip comprising the imaging system, a control chip comprising the control circuitry, an optical source chip comprising the optical source generating circuitry, and projection system chip comprising the projection system, wherein the chips are arranged in a chip stack. The chips may be made using semiconductor fabrication materials and techniques, including but not limited to Si, InP, GaAs, Liquid Crystal materials, and BGA/C4/micro-BGA, through substrate (or silicon) vias (TSVs), micro-TSVs, and solder or oxide bonding techniques.

In an embodiment, a device according to any of the aspects above comprises a wireless communication antenna (e.g., for receiving programming signals) and/or an inductive coupling coil (e.g., for wireless charging) embedded in the material of the body.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 shows a diagram of a healthy eye.

FIG. 2 shows a diagram of a damaged eye.

FIG. 3A shows a diagram of a healthy retina corresponding to the eye of FIG. 1.

FIG. 3B shows a diagram of a damaged retina corresponding to the eye of FIG. 2.

FIG. 4A shows a diagram of an image that is incident on the healthy retina of FIG. 3A and the resulting view to the person.

FIG. 4B shows a diagram of an image that is incident on the damaged retina of FIG. 3B and the resulting view to the person.

FIG. 5 shows an embodiment of a device implanted in a capsular bag of an eye in accordance with aspects of the invention.

FIG. 6 shows an embodiment of a device implanted in a ciliary sulcus of an eye in accordance with aspects of the invention.

FIG. 7 shows an embodiment of a device implanted in an anterior chamber of an eye in accordance with aspects of the invention.

FIG. 8 shows a diagram of an exemplary projection of an image on a retina by a device in accordance with aspects of the invention.

FIG. 9 shows a diagram of an exemplary projection of an image on a retina by a device in accordance with aspects of the invention.

FIG. 10 shows a diagram of an image projected on the healthy area of the retina by a device in accordance with aspects of the invention, and a view of what the person sees based on the image being projected in the manner shown.

FIG. 11 shows an exemplary point-to-point pixelated mapping from a 2D directionally programable optical array to a retina optical nerve surface in accordance with aspects of the invention.

FIG. 12 shows an exemplary point-to-point pixelated mapping from a 2D directionally programable optical array to a retina optical nerve surface in accordance with aspects of the invention.

FIG. 13 shows a flowchart of an exemplary method in accordance with aspects of the invention.

FIG. 14A shows exemplary locations on eyeglasses for coils that may be used to wirelessly charge a device in accordance with aspects of the invention.

FIG. 14B shows an exemplary location on an eyepatch for coils that may be used to wirelessly charge a device in accordance with aspects of the invention.

FIGS. 15A and 15B show embodiments of an OPA device in accordance with aspects of the invention.

FIG. 16 shows an embodiment of a microlens device in accordance with aspects of the invention.

FIG. 17A shows a flowchart of an exemplary method in accordance with aspects of the invention.

FIG. 17B shows a coarse grid used in the method of FIG. 17A.

FIG. 17C shows a fine grid used in the method of FIG. 17A.

FIGS. 18A and 18B show a side view and a top view, respectively, of a device in accordance with aspects of the invention.

FIGS. 19A-D show a device in accordance with aspects of the invention.

FIGS. 20A-D show a device in accordance with aspects of the invention.

FIGS. 21A-E show a device in accordance with aspects of the invention.

FIG. 22 shows a device in accordance with aspects of the invention.

FIG. 23 shows a device in accordance with aspects of the invention.

FIG. 24 shows devices in accordance with aspects of the invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The present invention relates generally to ocular implants and, more particularly, to electronic surgical eye implants having smart haptics that can be used in both therapeutic and diagnostic applications. To be compatible with a minimally invasive surgery, the surgical incision for placing an electronic eye implant should be as small as possible. The present disclosure describes a foldable, flexible, or collapsible “smart” haptics for an electronic eye implant. The smart haptics in this disclosure are described with respect to an electronic eye implant but can be used with other devices. Putting an expandable eye implant into the surgical incision in the eye is a bit like building a ship in a bottle; it goes in with a small cross-section and is then expanded into place in the eye. To facilitate minimally invasive insertion and removal, the present disclosure describes implants having smart haptics that are designed to be collapsible for insertion and extraction through a small cross-section incision in the eye.

Embodiments described herein include a foldable/collapsible structure allowing smart haptics to fit within a small incision in the eye cross-sectional area and later being expanded to a larger size. Embodiments described herein include a rollable or foldable/flexible substrate holding multiple components such as chips, batteries, capacitors, antennas, etc.

Embodiments described herein include a smart/electronic intraocular lens (IOL) that consists of bio-compatible optically transparent/translucent material that is flexible enough to be folded or rolled into a syringe for implantation into the eye. To enable a smart/electronic IOL, these IOLs, to be easily surgically implanted, are collapsible and expandable such as a by rolling, folding, etc. In embodiments, electronic components in the haptics, such as charging coils and antennas, are arranged in the IOL to have a sufficient shape (e.g., number of coils, etc.) and to be sufficiently flexible such that they maintain their electrical continuity throughout configuration changes of the IOL from an expanded configuration to a collapsed configuration and back to the expanded configuration. In embodiments, other electronic components in the IOL (e.g., chips, batteries, etc.) as well as the electronic components in the haptics are arranged such that they allow this collapsing and expanding to provide for insertion into and subsequent unfurling in the eye.

In some embodiments, the IOL comprises opaque materials and need not be transparent as with a conventional polymethylmethacrylate (PMMA) IOL. In this manner, embodiments may have different elasticity and optical/thermal properties compared to conventional IOLs.

In embodiments, the alignment of electronic components and chips in the IOL can be such that they better facilitate foldability and rollability, e.g., such that their positioning on the flexible substrate is more amenable to rolling into a syringe.

In embodiments, a device comprises a projection system, preferably an optical array, more preferably an optical phased array (OPA) or a microlens array, integrated to control electronics and charged-coupled device (CCD)/electronic cameras. In embodiments, a camera is integrated in a single assembly with the implanted projection system. In this way, when the device is implanted in an eye of a patient, the patient has vision which tracks with eyeball direction as opposed to, for example, a camera system mounted on a pair of glasses and communicated to the projection system from a wired/tethered or wireless network bridge.

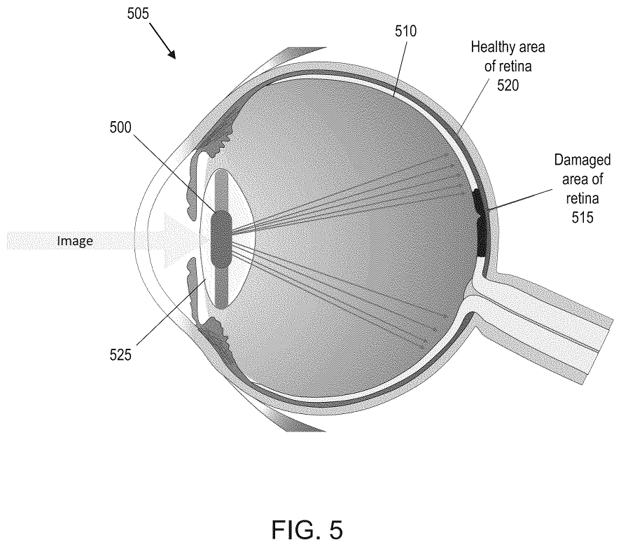

In an embodiment, the camera, optical signal sources, control electronics, programmable projection system, and power source (e.g., batteries) are all integrated in one device which is surgically implanted in the eye as shown. Exemplary embodiments of implants are shown in FIGS. 5-7.

In embodiments, the surgically implanted chip is wirelessly powered via an inductively coupled primary coil that can be positioned at various locations near the implanted chip, such as for example, on a pair of glasses or on a monocle-style mounting.

Devices according to aspects of the invention allow very detailed (e.g., 1 μm to 50 μm spot size) visible light probing of the retina, including the extreme periphery of functioning retinal tissue. An optical phased array (OPA) implementation of the optical array is well-suited for this application because it has good spot size control and no moving parts.

In embodiments, very detailed (e.g., micron-scaled) maps of functional and non-functional areas of the retina are made by probing/testing precise areas of the retina using an implant in accordance with aspects of the invention.

By being able to probe/test precise areas of the retina, detailed, micron-scaled maps of the functional retina tissue can be created. This mapping provides an advantage over devices that do not utilize mapping, since the mapping permits the inventive devices to precisely target light onto functional areas of the retina. In embodiments, a device is implanted near the front of the eye. This type of surgery is much less invasive and problematic than trying to implant a chip with an array of electrical needle probes or chemical injection ports directly onto the retina surface. Embodiments thus provide a much more practical approach and will allow many more doctors to be able to be trained for the procedure which would be similar to other common surgical eye procedures/implants.

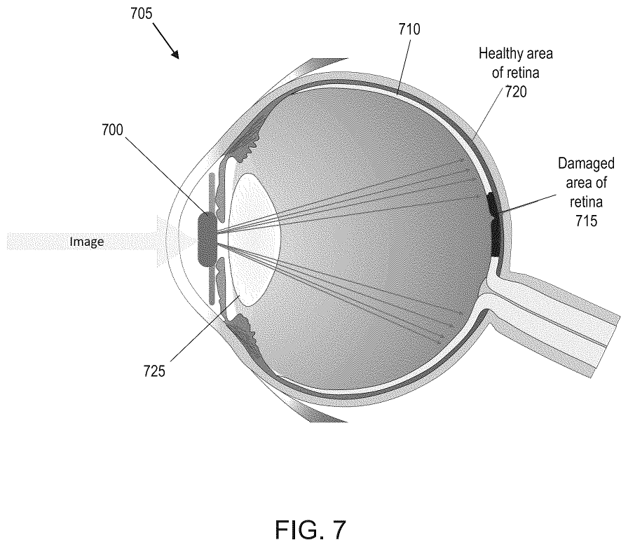

In one embodiment, a wirelessly powered and programmable device including an integrated CCD, control electronics, and projection system is surgically implanted in the eyeball as shown, for example, in FIG. 7. In one example, the device is hermetically sealed and completely self-contained.

Devices according to aspects of the invention may be used diagnostically, e.g., for creating detailed functional retinal tissue maps. Devices according to aspects of the invention may be used therapeutically, e.g., for image construction and projection onto functional retinal tissue in real time.

In embodiments, there is a surgically implanted integrated device that includes a camera, control electronics, programmable circuitry, and a projection system for retinal image generation. In embodiments, the device is used for mapping healthy (also called functional) retina tissue and unhealthy (also called damaged or non-functional) retina tissue. In embodiments, the device is used for image projection onto healthy retina tissue. In embodiments, the device is used to project eyeball-motion directed images selectively onto the healthy portions of retina tissue according to a map. The device may have wirelessly powered variants. The device may be used to perform a method of mapping healthy and unhealthy areas of the retina.

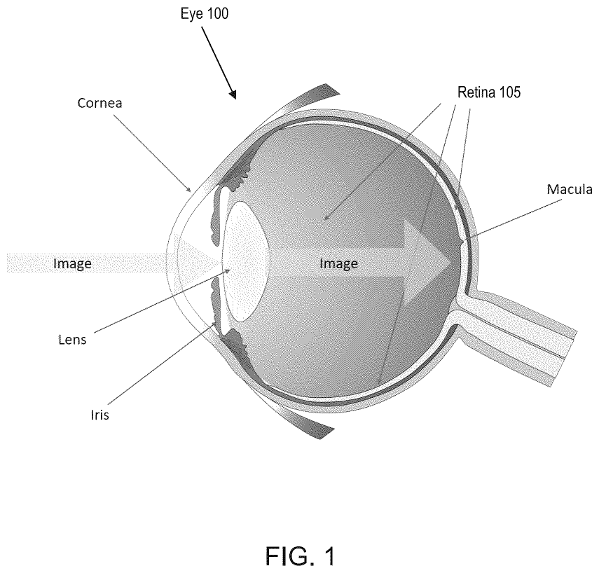

FIG. 1 shows a diagram of a healthy eye 100. As shown in FIG. 1, an image in the form of visible light enters the cornea and is focused onto the lens and then finally onto the macula e.g., (central portion of the retina 105) which allows for clear vision.

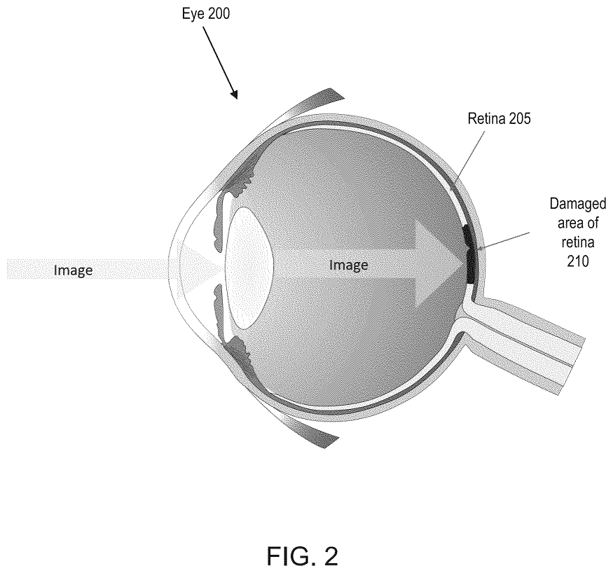

FIG. 2 shows a diagram of a damaged eye 200. As shown in FIG. 2, retinal scarring in the macula (e.g., macular degeneration) results in a damaged area 210 of the retina 205 that causes loss of central vision (e.g., scotoma). Implementations of the invention seek to take advantage of the still healthy areas of the retina, e.g., not including the damaged area, to help patients regain significant visual function.

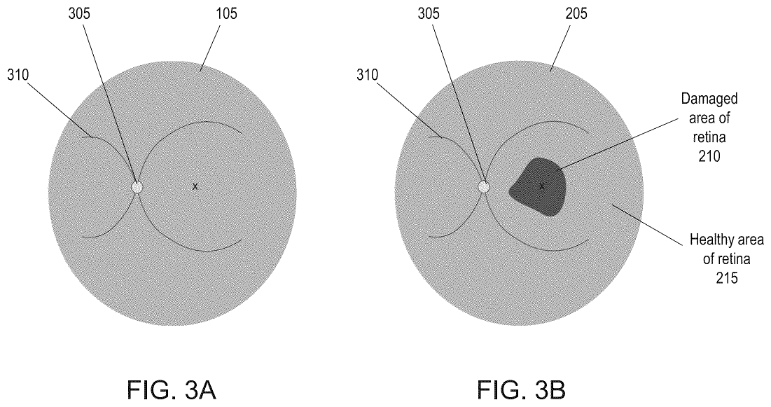

FIG. 3A shows a diagram of a healthy retina 105 corresponding to the eye 100 of FIG. 1. Also shown are the optic nerve/disc 305 and retinal veins/arteries 310. As shown in FIG. 3A, the retina 105 does not have a damaged portion and, thus, provides normal central vision for the person.

FIG. 3B shows a diagram of a damaged retina 205 corresponding to the eye 200 of FIG. 2. Also shown are the optic nerve/disc 305 and retinal veins/arteries 310. As shown in FIG. 3B, the retina 205 includes a damaged area 210 that produces a large central scotoma in the person's vision. As shown in FIG. 3B, the retina 205 includes undamaged area 215 around the damaged area 205.

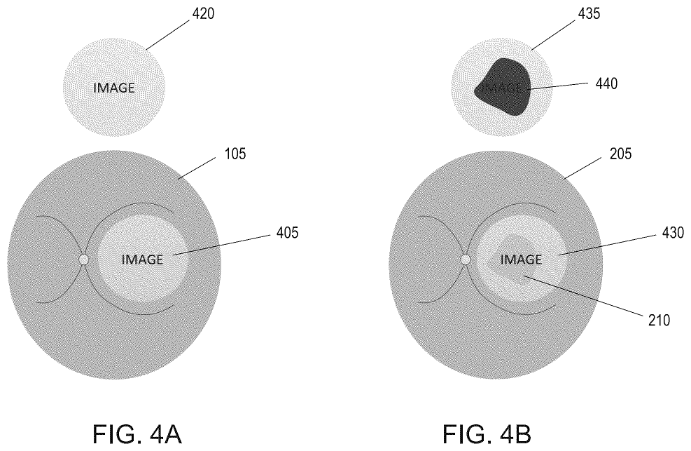

FIG. 4A shows a diagram of an image 405 (e.g., visible light) that is incident on the healthy retina 105 of FIG. 3A. FIG. 4A illustrates a view 420 of what this person sees based on the image 405 being incident on the retina 105. As shown in FIG. 4A, the view 420 of what this person sees is a normal view without any scotoma.

FIG. 4B shows a diagram of an image 430 (e.g., visible light) that is incident on the damaged retina 205 of FIG. 3B. FIG. 4B illustrates a view 435 of what this person sees based on the image 430 being incident on the retina 205 having the damaged area 210. As shown in FIG. 4B, the view 435 of what this person sees has a large central scotoma 440, represented in this case by a dark or fuzzy spot in an otherwise normal view.

FIG. 5 shows an embodiment of a device 500 implanted in a capsular bag of an eye in accordance with aspects of the invention. The device 500 may comprise an IOL. As shown in FIG. 5, the eye 505 includes a retina 510 that has a damaged area 515 and a healthy area 520, e.g., similar to the retina 205 shown in FIGS. 3B and 4B. The eye 505 also includes a capsular bag 525 which normally contains the lens, e.g., the human crystalline lens. In accordance with aspects of the invention, the human lens is removed and replaced with the device 500 that is configured to receive an image (e.g., visible light) from outside the eye and project the image onto the healthy area 520 of the retina 510 using beam steering provided by a projection system in the device 500. By projecting the image onto the healthy area 520 of the retina 510 and avoiding projecting the image onto the damaged area 515, the implanted device 500 provides this person with a view that eliminates or greatly reduces the scotoma that this person would otherwise have if the device 500 were not present.

The device 500 may be implanted in the capsular bag 525 after primary cataract surgery or as an intraocular lens exchange with intact posterior capsule. An exemplary method for implanting the device 500 in the capsular bag 525 includes: making a 6-8 mm incision at the limbus or slightly posterior (1-2 mm) posterior to the limbus; through a pharmacologically dilated pupil, making a 6-8 mm diameter opening in the anterior capsular bag; and removing the human crystalline lens entirely in an extra capsular fashion such as phacoemulsification. If the eye is pseudophakic with an intact posterior capsule, then intraocular lens is dissected free of its capsular attachment and removed from the eye. The capsular opening is then widened if necessary. The device 500 is then placed through the primary incision and into the capsular bag. The haptics of the device 500 keep the implant centered in the capsular bag as it heals and creates a fibrotic membrane to stabilize the implant, and place the device 500 directly in the visual axis for the purpose of projecting the central image onto the healthiest part of the retina as close to the damaged area 515 as possible. In embodiments where the device 500 has external wiring, the wires coming from the device 500 may be placed anterior to the anterior capsule and posterior to the iris and routed to the limbus, for example, through a 30 or 27 gauge temporal sclerotomy 2-3 mm posterior to the limbus. The wires may be left subconjunctival to prevent foreign body sensation. All support material may be removed, and the primary wound may be closed with sutures if needed. The device 500 is thus held inside the capsular bag 525. Over time, the bag fibrosis around the haptics of the implant is stable in place.

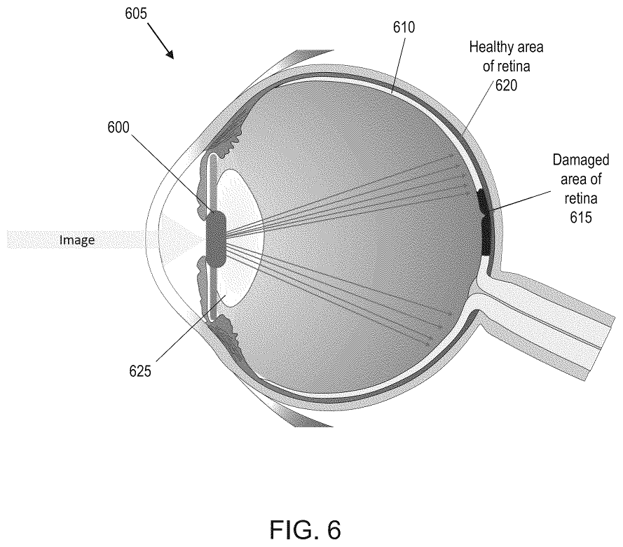

FIG. 6 shows an embodiment of a device 600 implanted in a ciliary sulcus of an eye in accordance with aspects of the invention. The device 600 may comprise an IOL. The ciliary sulcus is a small space between the posterior surface of the iris base and the anterior surface of the ciliary body. As shown in FIG. 6, the eye 605 includes a retina 610 that has a damaged area 615 and a healthy area 620, e.g., similar to the retina 205 shown in FIGS. 3B and 4B. The eye 605 also includes a capsular bag 625 which normally contains the lens, e.g., the human crystalline lens. In accordance with aspects of the invention, the human lens is removed and the device 600 is inserted into the ciliary sulcus. The device 600 is configured to receive an image (e.g., visible light) from outside the eye and project the image onto the healthy area 620 of the retina 610 using beam steering provided by a projection system in the device 600. By projecting the image onto the healthy area 620 of the retina 610 and avoiding projecting the image onto the damaged area 615, the implanted device 600 provides this person with a view that eliminates or greatly reduces the scotoma that this person would otherwise have if the device 600 were not present.

The device 600 may be implanted in the ciliary sulcus after primary cataract surgery with compromised posterior capsule or as an intraocular lens exchange with open posterior capsule. An exemplary method for implanting the device 600 in the ciliary sulcus includes: making a 6-8 mm incision at the limbus or slightly posterior (1-2 mm) posterior to the limbus; through a pharmacologically dilated pupil, making a 6-8 mm diameter opening in the anterior capsular bag; and removing the human crystalline lens entirely in an extra capsular fashion such as phacoemulsification. A thorough anterior vitrectomy is performed in the presence of a posterior capsule defect. If the eye is pseudophakic with an open posterior capsule, the intraocular lens is dissected free of its capsular attachment and removed from the eye. The capsular opening is then widened if necessary and a thorough anterior vitrectomy is performed. The device 600 is placed through the primary incision and into the ciliary sulcus on the anterior aspect of the capsular bag, directly posterior to the iris. The haptics of the device 600 will keep the implant centered in the ciliary sulcus to stabilize the implant and place the device 600 directly in the visual axis for the purpose of projecting the central image onto the healthiest part of the retina as close to the damaged area 615 as possible. In embodiments where the device 600 has external wiring, the wires coming from the device 600 may be placed anterior to the anterior capsule and posterior to the iris and routed to the limbus, for example, through a 30 or 27 gauge temporal sclerotomy 2-3 mm posterior to the limbus. The wires may be left subconjunctival to prevent foreign body sensation. All support material may be removed, and the primary wound may be closed with sutures if needed. The device 600 haptics rest in the ciliary sulcus posterior to the iris and directly anterior to the capsular bag, which stabilizes the lens.

FIG. 7 shows an embodiment of a device 700 implanted in an anterior chamber of an eye in accordance with aspects of the invention. The device 700 may comprise an IOL. As shown in FIG. 7, the eye 705 includes a retina 710 that has a damaged area 715 and a healthy area 720, e.g., similar to the retina 205 shown in FIGS. 3B and 4B. The eye 705 also includes a capsular bag 725 which normally contains the lens, e.g., the human crystalline lens. In accordance with aspects of the invention, the human lens is removed and the device 700 is inserted into the anterior chamber of an eye, e.g., anterior to the iris. The device 700 is configured to receive an image (e.g., visible light) from outside the eye and project the image onto the healthy area 720 of the retina 710 using beam steering provided by a projection system in the device 700. By projecting the image onto the healthy area 720 of the retina 710 and avoiding projecting the image onto the damaged area 715, the implanted device 700 provides this person with a view that eliminates or greatly reduces the scotoma that this person would otherwise have if the device 700 were not present.

The device 700 may be implanted in the anterior chamber after primary cataract surgery with no capsular support or as an intraocular lens exchange with no capsular support. An exemplary method for implanting the device 700 in the anterior chamber includes: making a 6-8 mm incision at the limbus or slightly posterior (1-2 mm) posterior to the limbus; through a pharmacologically dilated pupil, making a 6-8 mm diameter opening in the anterior capsular bag; and removing the human crystalline lens entirely in an extra capsular fashion such as phacoemulsification. A thorough anterior vitrectomy is performed in the absence of sufficient capsular support. If the eye is pseudophakic with an open posterior capsule, the intraocular lens is dissected free of its capsular attachment and removed from the eye, and a thorough anterior vitrectomy is performed in the absence of sufficient capsular support. Miosis of the pupil may be performed to provide support for the device 700. The device 700 is then placed through the primary incision and into the anterior chamber directly anterior to the iris. The haptics of the device 700 are seated into the anterior chamber angle to stabilize the implant and place the device 700 directly in the visual axis for the purpose of projecting the central image onto the healthiest part of the retina as close to the damaged area 715 as possible. A small peripheral iridotomy may be performed to prevent pupillary block. In embodiments where the device 700 has external wiring, the wires coming from the device 700 may be placed anterior to the anterior capsule and posterior to the iris and routed to the limbus, for example, through a 30 or 27 gauge temporal sclerotomy 2-3 mm posterior to the limbus. The wires may be left subconjunctival to prevent foreign body sensation. All support material may be removed, and the primary wound may be closed with sutures if needed.

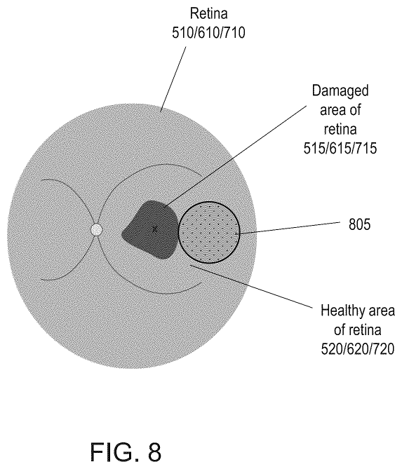

FIG. 8 shows a diagram of an exemplary projection of an image on a retina 510/610/710 by a device 500/600/700 in accordance with aspects of the invention. As described with respect to FIGS. 5-7, the device 500/600/700 receives an incoming image, in the form of visible light from outside the eye, and projects the image onto a healthy area 520/620/720 of the retina while avoiding projecting the image onto the damaged area 515/615/715. FIG. 8 shows the projection area 805 relative to the damaged area 515/615/715. The shape of the projection area 805 in FIG. 8 is illustrative, and the projection area 805 may have other shapes different than what is shown in FIG. 8.

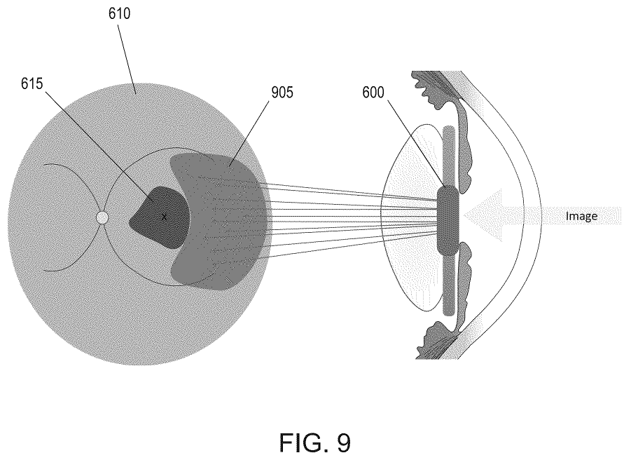

FIG. 9 shows a diagram of an exemplary projection of an image on a retina by an a device 600 in accordance with aspects of the invention. As described with respect to FIG. 6, the device 600 receives an incoming image, in the form of visible light from outside the eye, and projects the image onto a healthy area of the retina while avoiding projecting the image onto the damaged area 615. FIG. 9 shows the projection area 905 relative to the damaged area 615. Specifically, the device 600 takes the central image and shifts the projection onto the adjacent healthy area of the retina. In this manner, the healthy area of the retina adjacent to the damaged area of the retina can be used for central vision. The shape of the projection area 905 in FIG. 9 is illustrative, and the projection area 905 may have other shapes different than what is shown in FIG. 9. Although FIG. 9 only shows the device 600, it should be understood that the device 500 and the device 700 may function in a similar manner, with a difference being where the different devices 500, 600,700 are implanted in the eye.

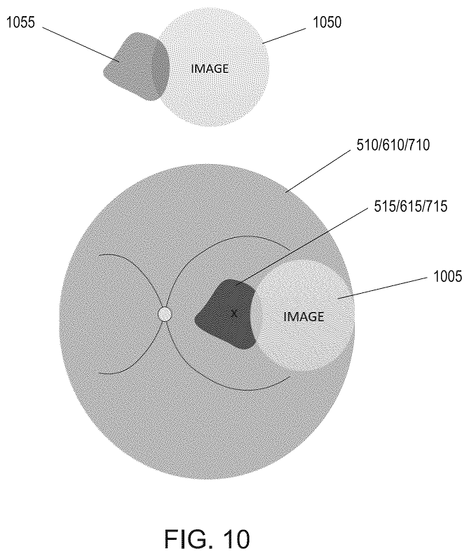

FIG. 10 shows a diagram of an image 1005 projected on the healthy area of the retina 510/610/710 by a device 500/600/700 in accordance with aspects of the invention, and a view 1050 of what the person sees based on the image being projected in the manner shown. As described herein, the device 500/600/700 projects the image onto the healthy area of the retina adjacent to the damaged area 515/615/715 of the retina. In this manner, the view 1050 of what the person sees has the scotoma 1055 shifted away from the center, such that the person can now see central visual details unimpeded by the scotoma. Comparing the view 1050 of FIG. 10 to the view 435 of FIG. 4B, it is evident that the device 500/600/700 provides a vast improvement in central vision for the person.

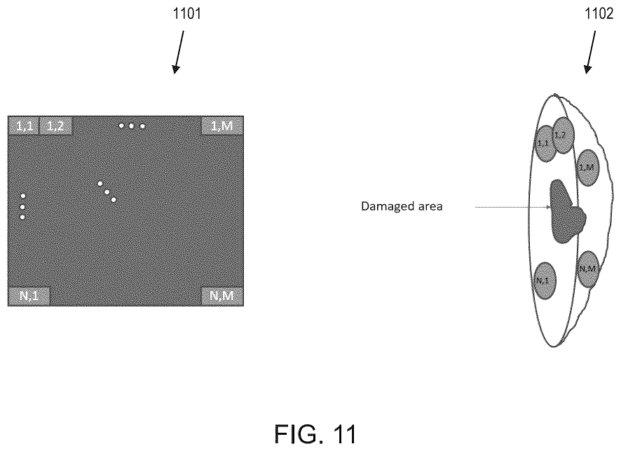

FIG. 11 shows an exemplary point-to-point pixelated mapping from a 2D directionally programable optical array 1101 to a retina 1102 optical nerve surface in accordance with aspects of the invention. In embodiments, individual elements of the array 1101 are mapped to locations on the retina 1102. The device 500/600/700 may use the mapping defined in the projection system (e.g., in an array of phased array emitters or microlenses) to control the beam steering to project the image onto healthy areas of the retina and avoid projecting onto the damaged areas of the retina.

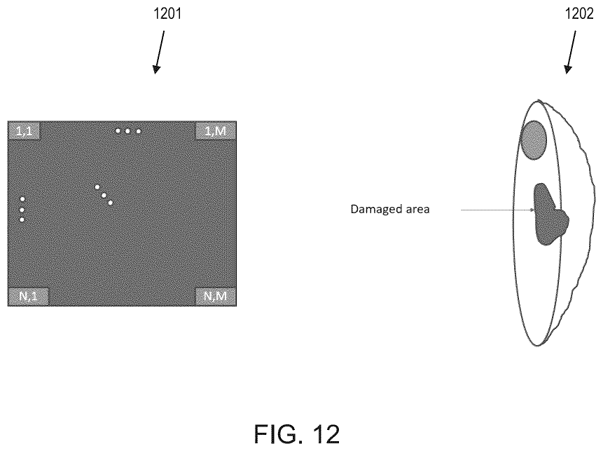

FIG. 12 shows an exemplary point-to-point pixelated mapping from a 2D directionally programable optical array 1201 to a retina 1202 optical nerve surface in accordance with aspects of the invention. In embodiments, the device 500/600/700 produces a moving spot that is dynamically swept across retina. Optical phased arrays operating in quasi near field regime (e.g., within a few wavelengths of the array surface) with spot sizes on the order of 10 μm are achievable. This spot size can be used to create high-definition quality pixel sizes on the retina. The visible wavelength is between about 380 nm and 750 nm and the eyeball is about 1 to 2 inches long, which is about 25 mm to 50 mm, which is about 30 to 130 wavelengths long, which means that the image projected by the device 500/600/700 can be close to the near field.

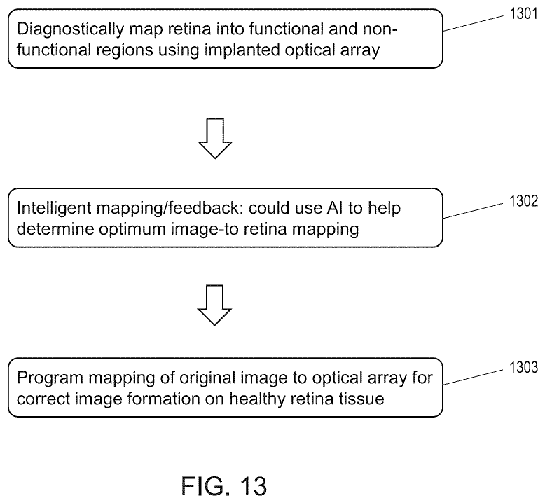

FIG. 13 shows a flowchart of an exemplary method in accordance with aspects of the invention. At step 1301, the implanted device 500/600/700 is used to diagnostically map a retina (of the eye in which the device is implanted) into functional and non-functional regions, e.g., healthy areas and damaged areas. This may include projecting an image onto a mapped location on the retina and receiving feedback from the person as to whether they can or cannot see the image clearly. This is repeated for all locations on the 2D array that are mapped to locations on the retina. In this manner, the implanted device 500/600/700 can be used to map the areas of the retina.

Step 1302 comprises using artificial intelligence to optimize the mapping that was determined at step 1301. The shape of the damaged areas and healthy areas of each person's retina will be unique and irregular. In embodiments, an optimum mapping of a regular 2D grid array of input pixels to the irregular healthy regions is determined using artificial intelligence. For example, an artificial neural network may be used to optimize a map of the regular input pixel grid to the irregular healthy retinal tissue, while minimizing the radius from the center of the retina, and while also seeking to maximize the symmetry of the pixel projection around the center. These sorts of constrained mapping tasks are well suited for AI in general and artificial neural networks specifically. The mapping here may take into account complex procedures using artificial neural networks that not only map to healthy retina tissue, but also take into account brain plasticity for image reconstruction.

Step 1303 involves program mapping of an original image to the optical array for correct image formation on the healthy area of the retina. In embodiments, the array that defines the mapping is stored in a programmable circuit of the device 500/600/700. In OPA embodiments, when in use, the device 500/600/700 uses the mapping defined in the array to control the phase shifting of the OPA elements to cause beam steering that projects the image onto the healthy areas of the retina as defined in the mapping.

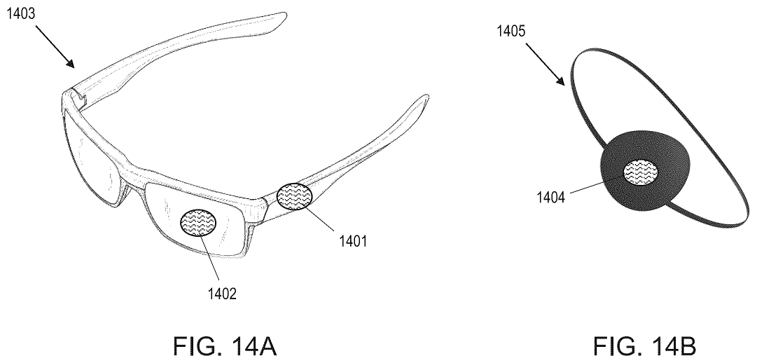

FIG. 14A shows exemplary locations 1401, 1402 on eyeglasses 1403 for coils that may be used to wirelessly charge the device 500/600/700 that is implanted in a person's eye. FIG. 14B shows an example of a location 1404 on an eyepatch 1405 for coils that may be used to wirelessly charge the device 500/600/700 that is implanted in a person's eye. The external charging system is not limited to eyeglasses or an eye patch, and can be on other devices, such as a contact lens. The external charging system itself can be rechargeable. For example, a contact lens may comprise a battery that is wirelessly rechargeable, e.g., from a docking station, and that same contact lens can include control circuity and charging coils that utilize power from the battery in the contact lens to inductively charge the device 500/600/700 when the contact lens is within range of the device.

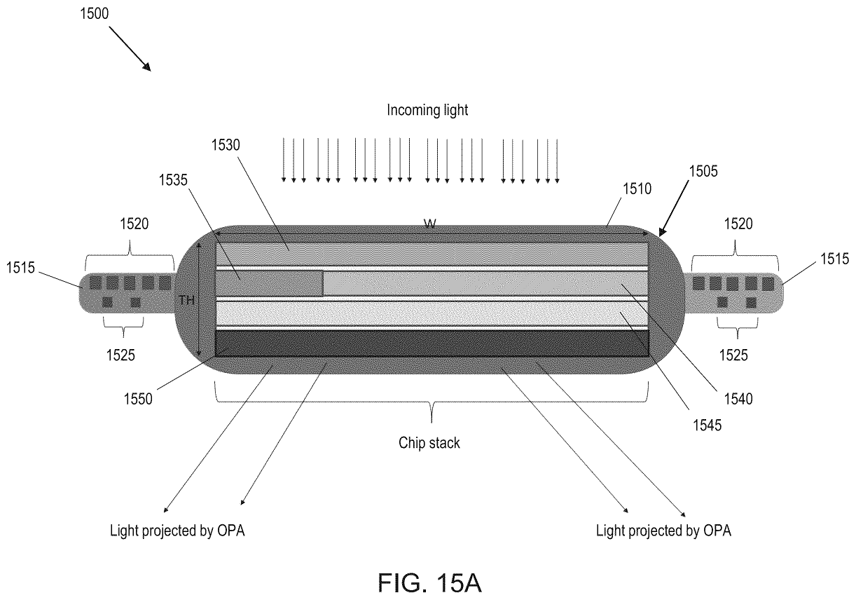

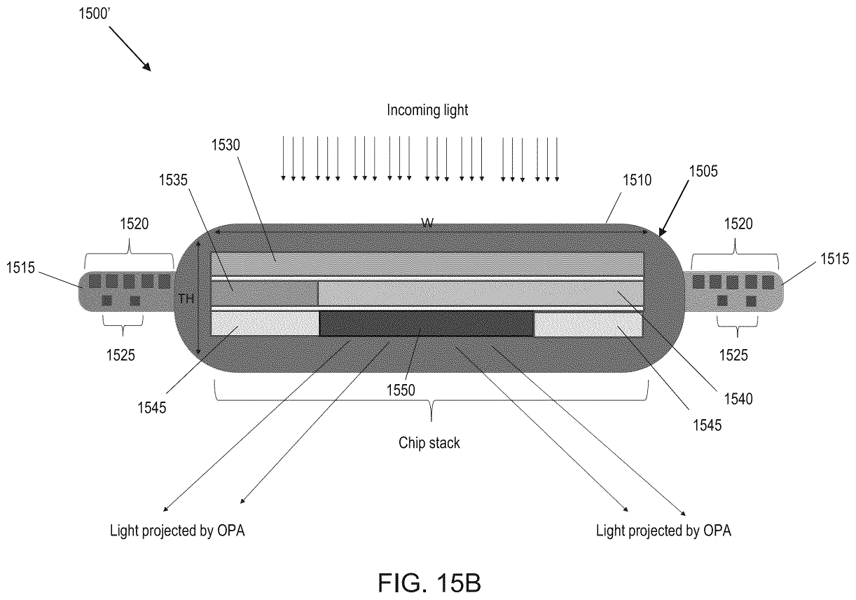

FIG. 15A shows an embodiment of an OPA device 1500 in accordance with aspects of the invention. The OPA device 1500 may be used as the devices 500/600/700 described herein. In embodiments, the OPA device 1500 includes a body 1505 that has a central portion 1510 and haptics 1515. The body 1505 may be made in the form of a single piece lens composed of materials such as acrylic and/or silicon lens material. In embodiments, the body 1505 comprises two haptics 1515 in the form of wings or tabs that each extend outward from the central portion 1510.

In embodiments, the OPA device 1500 comprises inductive coupling coils 1520, a wireless communication antenna 1525, an imaging system 1530, a power source 1535, control circuitry 1540, optical source generation circuitry 1545, and an optical phased array (OPA) 1550. In embodiments, the inductive coupling coils 1520 and wireless communication antenna 1525 are embedded in one or both haptics 1515, and the remaining elements 1530, 1535, 1540, 1545, 1550 are integrated in chip stack contained in the body 1505. As shown in FIG. 15A, the imaging system 1530 is at a first side of the chip stack such that it can receive incoming light from outside the eye, and the OPA 1550 is at a second side of the chip stack opposite the first side of the chip stack such that the OPA 1550 can project an image onto the retina inside the eye in which the OPA 1500 device is implanted. In embodiments, the imaging system 1530 receives incoming light from outside the eye and provides input to the control circuitry 1540 based on the received light, and the control circuitry 1540 provides electronic control signals to the optical source generation circuitry 1545 and the OPA 1550 based on the input received from the imaging system 1530. In this manner, a projection system comprising the OPA 1550 is controlled to reproduce an image received by the imaging system 1530 via projection onto the mapped areas of the retina.

FIG. 15A shows an embodiment of the OPA device 1500 in which the imaging system 1530, power source 1535, control circuitry 1540, optical source generation circuitry 1545, and OPA 1550 are arranged in four layers of a chip stack. FIG. 15B shows an embodiment of the OPA device 1500′ in which the imaging system 1530, power source 1535, control circuitry 1540, optical source generation circuitry 1545, and OPA 1550 are arranged in three layers of a chip stack. Other arrangements of these elements in a chip or chip stack may be used so long as the imaging system 1530 is positioned to receive incoming light and the OPA 1550 is positioned to project an image onto the retina inside the eye in which the OPA device is implanted.

The following description of the OPA device applies to both the OPA device 1500 of FIG. 15A and the OPA device 1500′ of FIG. 15B unless indicated otherwise. The imaging system 1530 may comprise a CCD/imaging chip. The power source 1535 may comprise a battery that is rechargeable either via wired connection or wirelessly. The control circuity 1540 may comprise a CMOS/analog/OPA control/wireless chip that is configured to control operation of the OPA device. The optical source generation circuitry 1545 may comprise an optical source/generation chip. The OPA 1550 may comprise components of an on-chip optical phase array including but not limited to: splitters, waveguides, phase shifters, amplifiers, and emitting elements.

The OPA device 1500/1500′ may be composed of sub-circuits which may be on disparate chip materials and made with disparate technologies, such as Si, InP, GaAs, Liquid Crystal, etc. This integrated system can be stacked in as shown in FIGS. 15A and 15B, with the connections between circuit elements being formed using BGA/C4/micro-BGA, through substrate (or silicon) vias (TSVs), and micro-TSVs. Physical connections between layers can be through solder or oxide bonding techniques.

In the OPA device 1500/1500′, sub-circuit chips may be thinned using wafer thinning techniques to be thin enough such that the entire system is such that the thickness dimension TH satisfies the expression 1 mm<=TH<=3 mm. These techniques are employed in stacked memory chips with wafers thinned to less than 20 μm thick and bonded to other wafers and connecting micro-TSVs are made between active layers that are 10 μm to 20 μm tall. The OPA device 1500/1500′ may be constructed such that the width dimension W satisfies the expression 1 mm<=W<=10 mm. An OPA device having these dimensions TH and W is suitable for implant in an eye, such as shown at FIGS. 5-7.

In the OPA device 1500/1500′, each sub-circuit system may be made with a different material technology and may be aligned and integrated such that they are on the same level as shown in the case of the optical source generation circuitry 1545 and the OPA 1550 being in a same layer in FIG. 15B. Additionally, the OPA 1550 may comprise integrated subcomponents such as SOI chips and Liquid Crystal cavities acting as voltage controlled optical phase shifters.

In the OPA device 1500/1500′, the control circuitry 1540 may contain wireless communication circuitry such that the integrated system could be programmed externally. In embodiments, once the image mapping to healthy retinal tissue is programmed, the device does not need any wireless communication to produce a retinal image in the healthy regions of the retina. The wireless communication antenna(s) for this system could be in the chips themselves (e.g., in the control circuitry 1540) or can be co-fabricated in the haptics as shown at elements 1525.

In embodiments, the power source 1535 comprises a rechargeable battery that can be wirelessly recharged through inductive coupling using the inductive coupling coils 1520 and an external charging coil, such as those illustrated in FIGS. 14A and 14B.

In embodiments, the OPA 1550 comprises an on-chip optical phased array that is capable of beam steering to project a visible light in a desired direction to create a projection of an image. On-chip optical phased arrays are understood by those of skill in the art, and any suitable fabrication may be used in implementations of the invention. For example, the optical phase shifters could use TiN micro heaters as actuators which would give KHz range responsivity, and are compact, but also have higher power consumption. They could also be PN-diode based charge injection phase shifters that use the carrier concentration near a PN interface to modulate optical index and therefore light (these have a responsivity in the MHz range, but they can be larger in size). The optical phase shifter could also be made of MOS actuators such that the charge concentration which modulates the optical index/phase is actuated by MOS cap carrier accumulation under an electrode (these can be larger in size, but are low power, and have responsivity in the GHz range). Further, the optical phase shifter could be fabricated with micro liquid crystal cavities, or through moving micro-mechanical systems (MEMs). The optical antennas of the phased array could be straight waveguide grating antennas, arc grating antennas, or different technologies in development such as hybrid plasmonic nano-antennas, which would be well-suited for rapidly controllable, 2D OPAs to maximize beam steering angle and optimizing quasi near-field beam formation on the retina nerve surface.

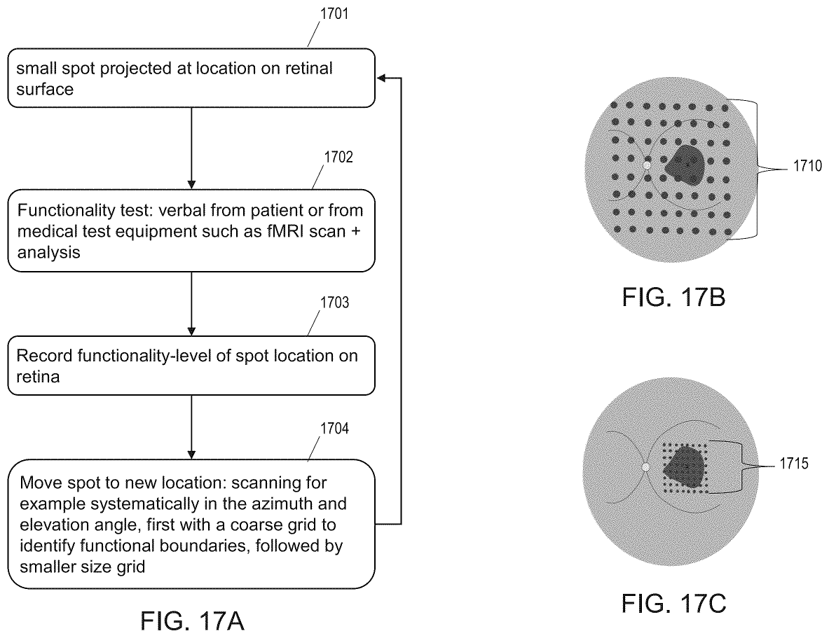

Still referring to FIGS. 15A and 15B, the imaging system 1530 may comprise any suitable type of on-chip imaging technology, such as a charge-coupled device (CCD). The imaging system 1530 may also include specialized local lens structures to enhance functionality of imaging chip. In embodiments, the output of the imaging system 1530 is a time-dependent electronic signal to control circuitry 1540. In embodiments, the control circuitry 1540 takes input of a time-dependent video signal, and an essentially fixed, but wirelessly programmable, mapping stored in memory (for example, in on-chip RAM) that is used to control where each pixel will be mapped to the retina surface. In embodiments, this programmable mapping is determined after diagnostic mapping as described, for example, at FIG. 17. During a programming phase, a wireless signal for the mapping is received through the wireless communication antenna 1525 which may be in the haptics and spiral around the center chipset as shown depicted in cross-section in FIGS. 15A and 15B.

In embodiments, the control circuitry 1540, once programmed with the desired mapping, controls the optical source generation circuitry 1545 with a time-dependent output that will control the phase shifters and optical source chips. In embodiments, the optical source generation circuitry 1545 can be located in a stack vertically separated from the OPA 1550 (e.g., as shown in FIG. 15A) or can be in a same level as the OPA 1550 (e.g., as shown in FIG. 15B). This may be done to maximize the coupling efficiency between the optical source and the waveguide chips, e.g., vertical beam input versus lateral beam input.

In embodiments, the optical source generation circuitry 1545 generates an output that is provided as an input to the OPA 1550, which may be an optical signal. In some embodiments, the optical signal in the visible wavelength range. In other embodiments, the optical signal is outside the visible wavelength range but is shifted to the visible wavelength range by the OPA 1550. In a particular example, the optical source generation circuitry 1545 provides a source of laser light for the OPA 1550. In this example, the optical source generation circuitry 1545 generates a laser beam source which is aligned and coupled into the OPA 1550 (e.g., either side-coupled or surface-coupled through grating waveguide couplers) from the optical source generation circuitry 1545 into the OPA 1550. The laser light coupled into the OPA 1550 is then routed using on-chip optical waveguides on the OPA 1550 to the various components of the OPA 1550 (e.g., phase shifters, optical antenna, etc.), where ultimately, it exits the OPA 1550 through an array of optical antennas which are targeting the image location points on the retina.

Still referring to FIGS. 15A and 15B, in an example embodiment, the OPA 1550 comprises an on-chip OPA that includes a splitter that splits the incoming optic signal into a plurality of optic signals, which are fed to a plurality of waveguides that are configured to carry optical signals in the visible wavelength range. The OPA 1550 can comprise a plurality of phase shifters that are coupled to respective ones of the plurality of waveguides, wherein the phase shifters are configured to shift the phase of the optical signals travelling in the waveguides. The OPA 1550 can comprise a plurality of emitters optically coupled to the plurality of waveguides, wherein a respective emitter receives an optical signal from its connected waveguide and outputs that optical signal by projecting the optical signal outward from the OPA 1550 (e.g., toward the retina). As is understood in the art, the outputs of plural emitters are combined through constructive and/or destructive interference to form a beam that is projected in a desired direction outward from the optical phased array, where the direction of the beam is controlled using the amount of phase shift applied at each of the wave guides. In this manner, the OPA 1550 may be used to output a beam of visible light in a desired direction toward the retina, e.g., to project an image on a healthy area of the retina as defined in the mapping.

With continued reference to FIGS. 15A and 15B, in embodiments the power source 1535 comprises an integrated battery component. Use of a battery is optional and provides the ability for the OPA device to operate for a time period without continuous inductive power coupling. In an alternative embodiment, the OPA device does not include a battery, and instead is powered continuously using inductive coupling. In the embodiments, one or more capacitors integrated into the chip assembly can be used to smooth the power supply from inductive charging coupling which may vary in strength versus time.

Still referring to FIGS. 15A and 15B, in embodiments the functionalities of each of the sub-component chips may be implemented with fewer layers, e.g., using only one or two larger substrates (1 to 10 mm in diameter) with several smaller chips attached. FIGS. 15A and 15B show cross-sections of particular implementations, but other integration/lateral/vertical chip attachments and placements are also contemplated.

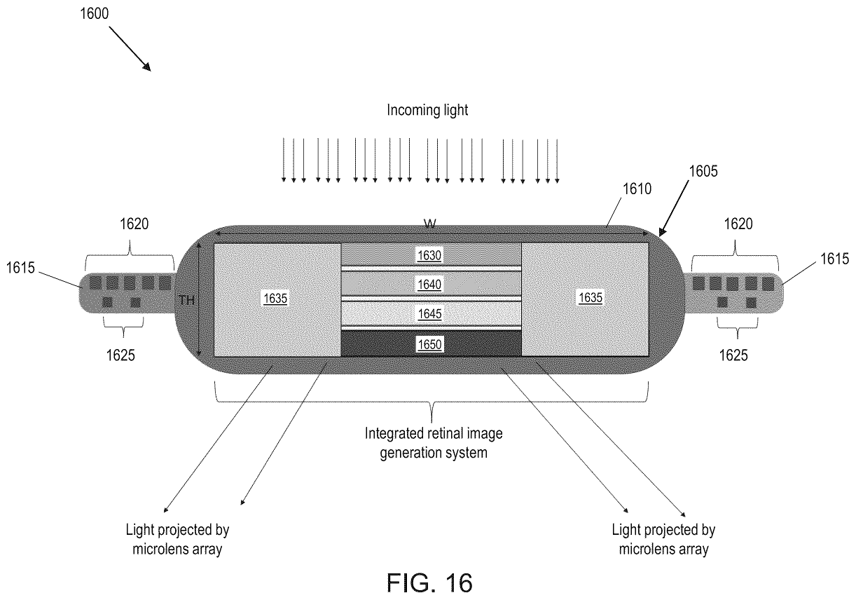

FIG. 16 shows an embodiment of a microlens device 1600 in accordance with aspects of the invention. The microlens device 1600 may be used as the devices 500/600/700 described herein. In embodiments, the microlens device 1600 includes a body 1605 that has a central portion 1610 and haptics 1615. The body 1605 may be made in the form of a single piece lens composed of materials such as acrylic and/or silicon lens material. In embodiments, the body 1605 comprises two haptics 1615 in the form of wings or tabs that each extend outward from the central portion 1610.

In embodiments, the microlens device 1600 comprises inductive coupling coils 1620, a wireless communication antenna 1625, an imaging system 1630, a power source 1635, control circuitry 1640, a light generation panel 1645, and a microlens array (MA) 1650. In an exemplary embodiment, the inductive coupling coils 1620 and wireless communication antenna 1625 are embedded in one or both haptics 1615, and the remaining elements 1630, 1635, 1640, 1645, 1650 are integrated in chip stack contained in the body 1605. As shown in FIG. 16, the imaging system 1630 is at a first side of the chip stack such that it can receive incoming light from outside the eye, and the MA 1650 is at a second side of the chip stack opposite the first side of the chip stack such that the MA 1650 can project an image onto the retina inside the eye in which the microlens device 1600 device is implanted. In embodiments, the imaging system 1630 receives incoming light from outside the eye and provides input to the control circuitry 1640 based on the received light, and the control circuitry 1640 provides electronic control signals to the light generation panel 1645 based on the input received from the imaging system 1630. Light emitted from the light generation panel 1645 can be steered after the MA 1650 directionally by switching which location source it originates on the panel 1645. In this way, a particular source location can be chosen and the projection direction from the light exiting the MA 1650 can be steered. In this manner, a projection system comprising the light generation panel 1645 and the MA 1650 is controlled to reproduce an image received by the imaging system 1530 via projection onto the mapped areas of the retina.

FIG. 16 shows an embodiment of the microlens device 1600 in which the imaging system 1630, power source 1635, control circuitry 1640, light generation panel 1645, and MA 1650 are arranged in four layers of a chip stack. However, embodiments are not limited to this configuration. Other arrangements of these elements in a chip or chip stack may be used so long as the imaging system 1630 is positioned to receive incoming light and the MA 1650 is positioned to project an image onto the retina inside the eye in which the microlens device is implanted.

The imaging system 1630 may comprise a CCD/imaging chip. The power source 1635 may comprise a battery that is rechargeable either via wired connection or wirelessly. The control circuity 1640 may comprise a CMOS/analog/light generation panel control/wireless chip that is configured to control operation of the microlens device 1600. The light generation panel 1645 may comprise a light source/generation chip. The MA 1650 may comprise components of a microlens array.

The microlens device 1600 may be composed of sub-circuits which may be on disparate chip materials and made with disparate technologies, such as Si, InP, GaAs, Liquid Crystal, etc. This integrated system can be stacked in as shown in FIG. 16, with the connections between circuit elements being formed using BGA/C4/micro-BGA, through substrate (or silicon) vias (TSVs), and micro-TSVs. Physical connections between layers can be through solder or oxide bonding techniques.

In the microlens device 1600, sub-circuit chips may be thinned using wafer thinning techniques to be thin enough such that the entire system is such that the thickness dimension TH satisfies the expression 1 mm<=TH<=3 mm. These techniques are employed in stacked memory chips with wafers thinned to less than 20 μm thick and bonded to other wafers and connecting micro-TSVs are made between active layers that are 10 μm to 20 μm tall. The microlens device 1600 may be constructed such that the width dimension W satisfies the expression 1 mm<=W<=10 mm. A microlens device having these dimensions TH and W is suitable for implant in an eye, such as shown at FIGS. 5-7.

In the microlens device 1600, each sub circuit system made with a different material technology may be aligned and integrated such that they are on a same level as shown in the case of the optical source chips and retinal image generation chip. Additionally, the retinal image generation chip itself may consist of integrated subcomponents such as SOI chips, Liquid Crystal cavities, LEDs, etc.

In the microlens device 1600, the control circuitry 1640 may contain wireless communication circuitry such that the integrated system could be programmed externally. In embodiments, once the image mapping to healthy retinal tissue is programmed, the device does not need any wireless communication to produce a retinal image in the healthy regions of the retina. The wireless communication antenna(s) for this system could be in the chips themselves (e.g., in the control circuitry 1640) or can be co-fabricated in the haptics as shown at elements 1625.

In embodiments, the power source 1635 comprises a rechargeable battery that can be wirelessly recharged through inductive coupling using the inductive coupling coils 1620 and an external charging coil, such as those illustrated in FIGS. 14A and 14B.

Still referring to FIG. 16, the imaging system 1630 may comprise any suitable type of on-chip imaging technology, such as a charge-coupled device (CCD). The imaging system 1630 may also include specialized local lens structures to enhance functionality of imaging chip. In embodiments, the output of the imaging system 1630 is a time-dependent electronic signal to control circuitry 1640. In embodiments, the control circuitry 1640 takes input of a time-dependent video signal, and an essentially fixed, but wirelessly programmable, mapping stored in memory (for example, in on-chip RAM) that is used to control where each pixel will be mapped to the retina surface. In embodiments, this programmable mapping is determined after diagnostic mapping as described, for example, at FIG. 17. During a programming phase, a wireless signal for the mapping is received through the wireless communication antenna 1625 which may be in the haptics and spiral around the center chipset as shown depicted in cross-section in FIG. 16. FIG. 17 shows a flowchart of an exemplary method in accordance with aspects of the invention. The method may be carried out using the any of the devices 500/600/700. At step 1701, the implanted device projects an image on a small spot on the retina. At step 1702, the person indicates (e.g., via verbal feedback) whether they can or cannot see the spot. At step 1703, the system records the indication (yes or no) and the settings of the device (e.g., the phase shifter settings that control the direction of the beam projected from the OPA). At step 1704, the device changes the settings to the change the direction of the projected beam to a different location on the retina. The method then returns to step 1701 to repeat the projecting (step 1701), receiving feedback (step 1702), and recording (step 1703). By following this method and marching through plural discrete directions of the beam, the system can be used to create the mapping of which directions of the beam point toward a healthy part of the retina and which directions of the beam point toward a damaged part of the retina. In embodiments, this scanning is performed initially using a coarse grid 1710 (e.g., as shown in FIG. 17B) and then using a smaller sized grid 1715 (e.g., as shown in FIG. 17C). In embodiments, the mapping determined using this method is programmed to the control circuitry of the device, e.g., using the wireless communication antenna as described herein. In embodiments, after being programmed, the mapping is used by the control circuitry of the device to control the projection system to form a beam in a desired direction onto a healthy area of the retina.

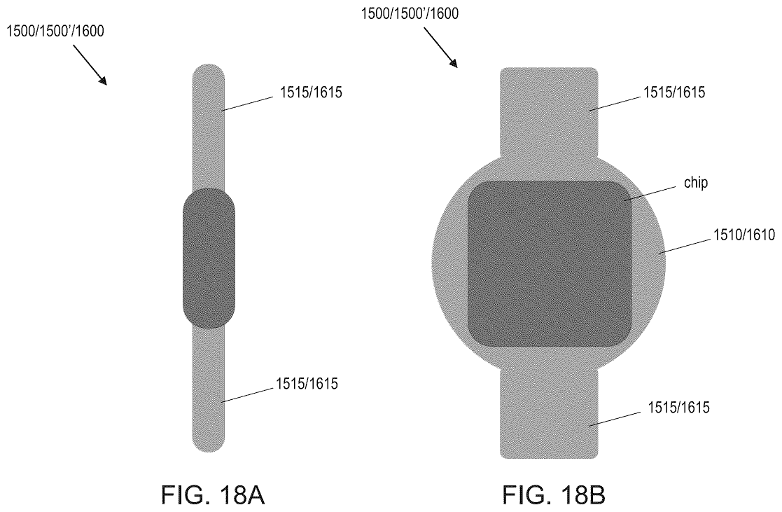

FIGS. 18A and 18B show a side view and a top view, respectively, of the device 1500/1500′/1600. As shown in FIGS. 18A and 18B, the device 1500/1500′/1600 includes a body comprising a center portion 1510/1610 and haptics 1515/1615. A chip (e.g., a stacked chip structure) containing the on-chip elements is disposed in the center portion 1510/1610. The body may be composed of acrylic and/or silicone lens material, e.g., to form a single piece implantable lens replacement. The haptics 1515/1615 allow for locating the device 1500/1500′/1600 in the center of the capsular bar or ciliary sulcus.

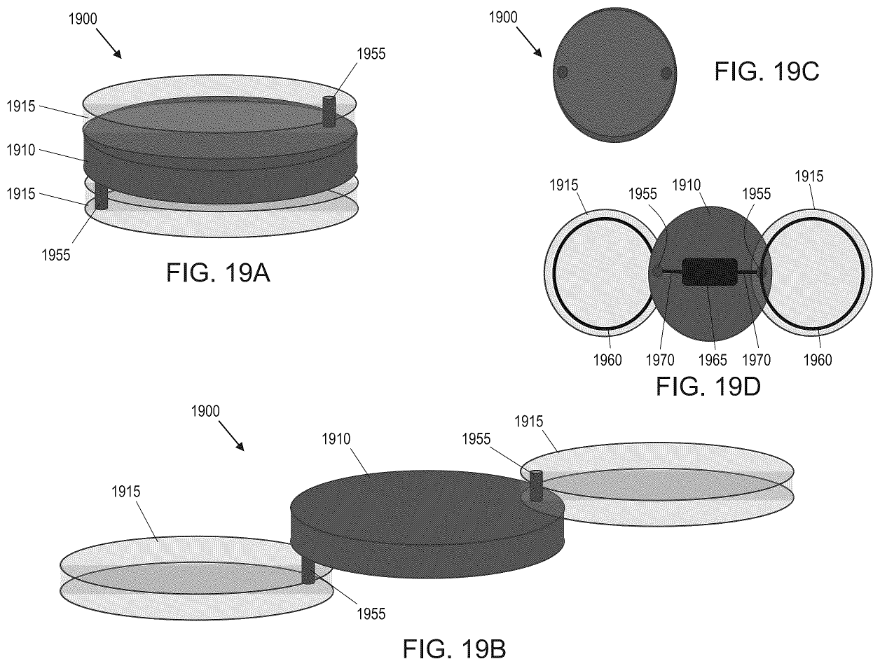

In accordance with aspects of the invention, the haptics (e.g., 1515/1615) of the device (e.g., 1500/1500′/1600) are movable relative to the center portion (e.g., 1510/1610) so that the device can be configured in a first, collapsed configuration for insertion into an eye. The collapsed configuration is useful for minimizing the size of the device when the device is being implanted in the eye, for example in the locations shown in FIGS. 5-7. In embodiments, after insertion into the eye while in the collapsed configuration, the haptics move relative to the main body so that the device is in a second, expanded configuration. The expanded configuration is useful for anchoring the device at a desired location in the eye after the device has been implanted in the eye. In accordance with aspects of the invention, one or more electronic components (e.g., inductive coupling coils 1520/1620 and/or wireless communication antenna 1525/1625) are embedded in the haptics and are designed to be sufficiently flexible to withstand movement of the haptics relative to the center portion such that these electrical elements are not damaged by such movement of the haptics relative to the center portion. In one example, the electronic components comprise flexible electrically conductive elements such as flexible wires or strips that maintain their electrical continuity when the device in the collapsed configuration, when the device is in the expanded configuration, and when the device is manipulated between the collapsed configuration and the expanded configuration and vice versa. In this manner, the electrically conductive elements remain intact and are useable for their intended purpose (e.g., inductive charging, wireless communication, etc.) when the device is in the expanded configuration in the eye after the device has been manipulated into the collapsed configuration during insertion into the eye. Different shapes of haptics containing electronic components and different types of movement of the haptics relative to the center portion are contemplated in the present disclosure, as illustrated by examples shown in FIGS. 19-23.

FIGS. 19A-D show a device 1900 in accordance with aspects of the invention. In embodiments, the device 1900 comprises an electronic surgical eye implant having smart haptics. The device 1900 may be used as any of the devices 500/600/700 shown in FIGS. 5-7 and may include elements described and shown in devices 1500/1500′/1600 of FIGS. 15-16. In embodiment, the device 1900 includes a central portion 1910 and haptics 1915 that moveably connected to the central portion 1910. In embodiments, the haptics 1915 are pivotally connected to the central portion 1910 by pivotal connection 1955, which may comprise a pin, hinge, or similar connection that provides for pivotal movement between two elements. As shown in FIGS. 19A and 19B, the pivotal connection permits the haptics 1915 to be moved relative to the central portion 1910 such that the device is in a first, collapsed configuration (FIG. 19A) or a second, expanded configuration (FIG. 19B). FIG. 19C shows a top-down view of the device 1900 in the collapsed configuration. FIG. 19D shows a top-down view of the device 1900 in the expanded configuration.

As shown in FIG. 19D, the device 1900 includes one or more electronic components 1960 in the haptics 1915. In embodiments, the electronic components 1960 comprise the inductive coupling coils 1520/1620 of FIGS. 15-16 and/or the wireless communication antenna 1525/1625 of FIGS. 15-16. In embodiments, the device 1900 includes an imaging and projection system 1965 in the central portion 1910. In embodiments, the imaging and projection system 1965 includes the imaging system 1530/1630 shown in FIGS. 15-16. In one example, the imaging and projection system 1965 comprises a projection system as shown in FIGS. 15A and 15B, including optical source generation circuitry 1545 and OPA 1550. In another example, the imaging and projection system 1965 comprises a projection system as shown in FIG. 16, including light generation panel 1645 and a MA 1650. In embodiments, the device 1900 includes other electronic components such as power source 1535/1635 of FIGS. 15-16 and control circuitry 1540/1640 of FIGS. 15-16, and these other electrical components may be located at any suitable locations in the device including the central portion 1910 or the haptics 1915. In embodiments, the device 1900 includes circuitry 1970 that electrically connects the electronic components 1960 to the other electronic components. The pivotal connection 1955 may be electrically conductive or have portions that are electrically conductive that electrically connect the electronic components 1960 to the circuitry 1970.

In use, the device 1900 may be arranged in the collapsed configuration shown in FIG. 19C and inserted through an incision in the eye. After insertion through the incision, the device 1900 may be arranged in the expanded configuration shown in FIG. 19D. While in the eye in the expanded configuration, the device 1900 may be wirelessly charged from a device outside the eye using the inductive coupling coils in the haptics 1915 (e.g., in the manner described at FIGS. 14A and 14B). While in the eye in the expanded configuration, the device 1900 may receive wireless data communication using the from a device outside the eye using the wireless communication antenna in the haptics 1915.

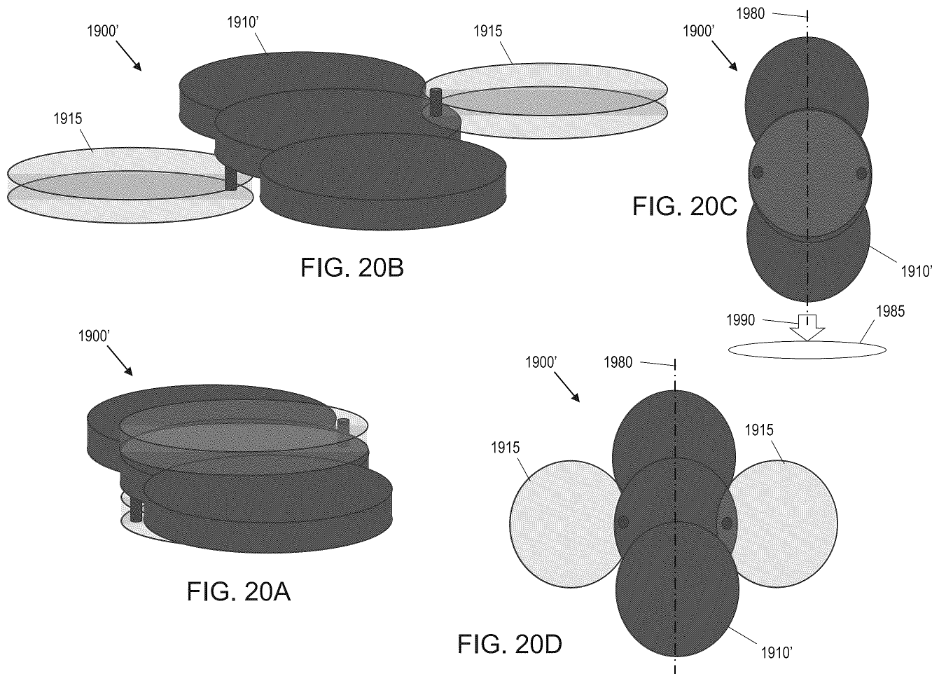

FIGS. 20A-D show an embodiment of the device 1900′ in accordance with aspects of the invention. Like device 1900, the device 1900′ comprises an electronic surgical eye implant having smart haptics. Like device 1900, the device 1900′ may be used as any of the devices 500/600/700 shown in FIGS. 5-7 and may include elements described and shown in devices 1500/1500′/1600 of FIGS. 15-16. Like device 1900, the device 1900′ includes a central portion 1910′ and haptics 1915 that moveably connected to the central portion 1910′. In embodiments, the device 1900′ is similar to the device 1900 in all ways, including electronic components, except that the central portion 1910′ of device 1900′ is shaped differently than the central portion 1910 of device 1900. As shown in FIGS. 20A-D, the central portion 1910′ of device 1900′ is elongated compared to the central portion 1910 of device 1900. In embodiments, the central portion 1910′ is elongated along an axis 1980 that is substantially aligned with a direction of insertion of the device 1900′ through an incision 1985 in the eye. The direction of insertion is shown with arrow 1990. In use, the device 1900′ may be placed in the collapsed configuration shown in FIG. 20C and inserted through the incision 1985. After insertion, the device 1900′ may be unfurled to the expanded configuration shown in FIG. 20D.

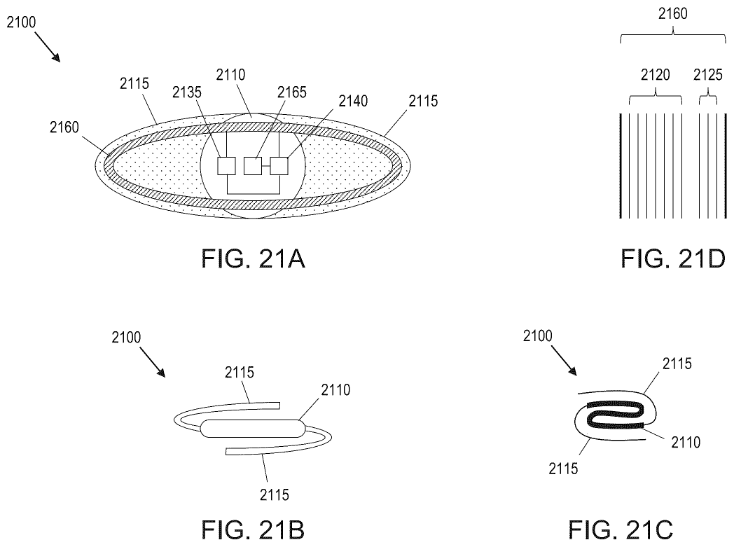

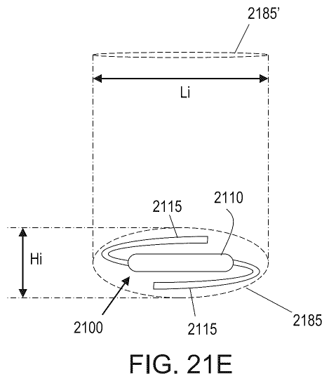

FIGS. 21A-B show a device 2100 in accordance with aspects of the invention. In embodiments, the device 2100 comprises an electronic surgical eye implant having smart haptics. The device 2100 may be used as any of the devices 500/600/700 shown in FIGS. 5-7. In embodiments, the device 2100 includes a central portion 2110 and haptics 2115 that are moveably connected to the central portion 2110. In embodiments, the haptics 2115 are flexibly connected to the central portion 2110, e.g., by one or more materials that permit flexure between the haptics 2115 and the central portion 2110.

FIG. 21A shows a top-down view of the device 2100 in an expanded configuration with the haptics 2115 not folded over the central portion 2110. FIG. 21B shows a side view of the device 2100 in a collapsed configuration with the haptics 2115 folded over the central portion 2110.

The device 2100 may include elements described and shown in devices 1500/1500′/1600 of FIGS. 15-16. In one example, the device 2100 includes a power source 2135, control circuitry 2140, one or more electronic components 2160 in the haptics 2115, and an imaging and projection system 2165 in the central portion 2110. In embodiments, the electronic components 2160 comprise the inductive coupling coils of FIGS. 15-16 and/or the wireless communication antenna of FIGS. 15-16. In embodiments, the imaging and projection system 2165 includes the imaging system of FIGS. 15-16. In one example, the imaging and projection system 2165 comprises a projection system as shown in FIGS. 15A and 15B, including optical source generation circuitry 1545 and OPA 1550. In another example, the imaging and projection system 2165 comprises a projection system as shown in FIG. 16, including light generation panel 1645 and a MA 1650.

FIG. 21A shows an example of the power source 2135 and the control circuitry 2140 being separate from (e.g., not integrated in a chip with) the imaging and projection system 2165. Although not shown, the power source 2135 and/or the control circuitry 2140 may be integrated with the imaging and projection system 2165, e.g., as shown in FIGS. 15-16. When not integrated with the imaging and projection system 2165, the power source 2135 may be in either the central portion 2110 or one of the haptics 2115. When not integrated with the imaging and projection system 2165, the control circuitry 2140 may be in either the central portion 2110 or one of the haptics 2115. The power source 2135 may be operatively connected to the control circuitry 2140 and the inductive coupling coils in the electronic components 2160, e.g., via electrically conductive wires or strips (shown but not numbered). The control circuitry may be operatively connected to the imaging and projection system 2165 and the wireless communication antenna in the electronic components 2160, e.g., via electrically conductive wires or strips (shown but not numbered). In this manner, the device 2100 may operate similar to devices 1500/1500′/1600 by receiving incoming light via an imaging system and projecting light onto a retina in a directed manner using the projection system.

FIG. 21B shows a side view of a first example of a collapsed configuration of the device 2100 in which the haptics 2115 are folded relative to the central portion 2110. In this example, the central portion 2110 is composed of a relatively rigid material (compared to the haptics 2115) such that the central portion 2110 substantially retains its shape when the haptics 2115 are folded.

FIG. 21C shows a side view of a second example of a collapsed configuration of the device 2100 in which the haptics 2115 are folded relative to the central portion 2110. In this example, the central portion 2110 and the haptics 2115 are both composed of a flexible material, such that both the central portion 2110 and the haptics 2115 are folded compared to the expanded configuration. The example shown in FIG. 21C is particularly useful when a pushing device, such as a syringe or plunger, is used to insert the device 2100 into the eye.

FIG. 21D shows a partial view of the electronic components 2160. In embodiments, the electronic components 2160 include one or more inductive charging coils 2120 that function similar to coils 1520/1620 described previously. In embodiments, the electronic components 2160 include one or more wireless communication antennas that function similar to antennas 1525/1625 described previously. The coils 2120 and antenna 2125 each comprise one or more flexible electrically conductive elements such as flexible wires or strips that maintain their electrical continuity when the device 2100 in the collapsed configuration, when the device 2100 is in the expanded configuration, and when the device 2100 is manipulated between the collapsed configuration and the expanded configuration and vice versa. In this manner, the electrically conductive elements remain intact and are useable for their intended purpose (e.g., inductive charging, wireless communication, etc.) when the device is in the expanded configuration in the eye after the device has been manipulated into the collapsed configuration during insertion into the eye.

As shown in FIG. 21A, the electronic components 2160 may be included in both the haptics 2115 and the central portion 2110. In embodiments, the flexible electrically conductive elements of the electronic components 2160 pass continuously through the haptics 2115 and the central portion 2110, such that the charging coil and/or antenna utilize almost the entire area of the device 2100 in the expanded configuration.

FIG. 21E shows the device 2100 in a collapsed configuration and relative to an incision 2185 in an eye. Number 2185 shows the incision in an open state with a length Li and a height Hi. Number 2185′ shows the same incision in a closed state.

In embodiments, a method comprises implanting the device 2100 into the eye. In embodiments, the implanting comprises: arranging the device 2100 in a collapsed configuration (e.g., as shown in FIGS. 21B or 21C); inserting the device 2100 into the eye (e.g., through incision 2185) while the device 2100 is in the collapsed configuration (e.g., as shown in FIG. 21E); and arranging the device 2100 in the expanded configuration (e.g., as shown in FIG. 21A) when the device is in the eye. In embodiments, the arranging the device in the collapsed configuration can be performed by physically manipulating the device. In one example, tweezers or a similar small-scale tool are used to fold the haptics over the central portion. In another example, a pusher tool, such as a syringe, is used. In one example, the device 2100 is loaded into a wide chamber of the syringe. When the plunger of the syringe is depressed, the device 2100 is pushed through a passage that decreases in size and causes the haptics (and possibly the central portion) to fold into the collapsed configuration. The distal end of the plunger is placed through the incision. When the device is pushed out of the distal end of the plunger, the device is inside the eye. In embodiments, the arranging the device 2100 in the expanded configuration when the device is in the eye can be performed by manipulating the device, e.g., with tweezers or other small-scale tools, while the device is in the eye. In another example, the device 2100 is constructed such that the haptics are biased toward their position in the expanded configuration, such that the device automatically arranges itself in the expanded configuration when it is placed in the eye. The biasing may be provided in any suitable manner, including by a biasing element embedded in the device, a biasing force created by an elasticity of the material due to the device being arranged in the collapsed configuration, a biasing force created by an interface between dissimilar materials, a biasing force created by different thicknesses of materials, etc. This method may be used with all the devices described herein.

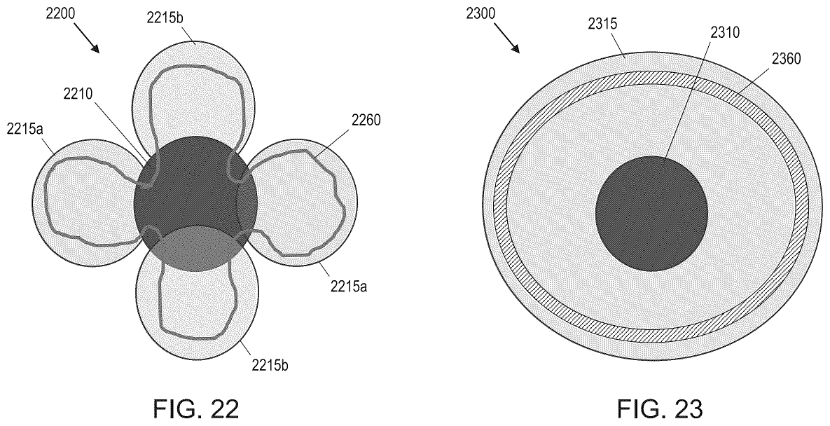

FIG. 22 shows a device 2200 in accordance with aspects of the invention. In embodiments, the device 2200 comprises an electronic surgical eye implant having smart haptics. The device 2200 may be used as any of the devices 500/600/700 shown in FIGS. 5-7. In embodiments, the device 2200 includes a central portion 2210 and haptics 2215a, 2215b that are moveably connected to the central portion 2210. In embodiments, the haptics 2215a, 2215b are flexibly connected to the central portion 2210, e.g., by one or more materials that permit flexure between the haptics 2215a, 2215b and the central portion 2210.

The device 2200 may be similar to device 2100 in that it includes a power source, control circuitry, one or more electronic components 2260 in the haptics, and an imaging and projection system in the central portion 2210, all of which operate in the same manner as those same elements in device 2100. The imaging and projection system are in the central portion 2210. The power source and control circuitry may be integrated with the imaging and projection system or may be separated from the imaging and projection system. As shown in FIG. 22, the one or more electronic components 2260 pass continuously through the multiple haptics 2215a, 2215b. Although four haptics 2215a, 2215b are shown in this example, implementations are not limited to this number and other numbers may be used.

In embodiments, the device 2200 is composed of materials that permit it to be arranged in the expanded configuration shown in FIG. 22 and also arranged in collapsed configurations such as those shown in FIGS. 21B and 21C. In one embodiment, the haptics 2215a and 2215b have a same degree of flexibility relative to the central portion 2210. In another embodiment, the haptics 2215a and 2215b have different degrees of flexibility relative to the central portion 2210. For example, both pairs of haptics 2215a and 2215b may be flexible, but one pair may be more flexible than the other.

FIG. 23 shows a device 2300 in accordance with aspects of the invention. In embodiments, the device 2300 comprises an electronic surgical eye implant having smart haptics. The device 2300 may be used as any of the devices 500/600/700 shown in FIGS. 5-7. In embodiments, the device 2300 includes a central portion 2310 and a haptics 2315 that is moveably connected to the central portion 2310. In embodiments, the haptic 2315 is flexibly connected to the central portion 2310, e.g., by one or more materials that permit flexure between the haptic 2315 and the central portion 2310.

The device 2300 may be similar to device 2100 in that it includes a power source, control circuitry, one or more electronic components 2360 in the haptics, and an imaging and projection system in the central portion 2310, all of which operate in the same manner as those same elements in device 2100. The imaging and projection system are in the central portion 2310. The power source and control circuitry may be integrated with the imaging and projection system or may be separated from the imaging and projection system. As shown in FIG. 23, the one or more electronic components 2260 pass continuously through the haptic 2315.

In embodiments, the device 2300 is composed of materials that permit it to be arranged in the expanded configuration shown in FIG. 23 and also arranged in collapsed configurations such as those shown in FIGS. 21B and 21C.

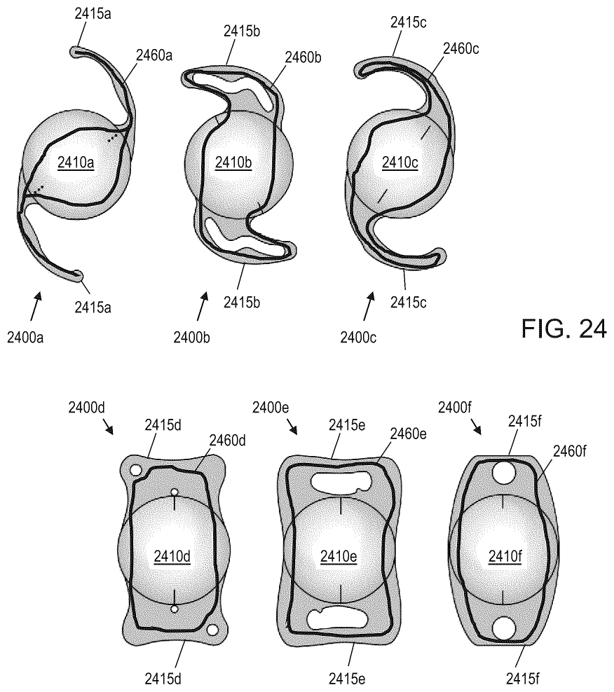

FIG. 24 shows examples of different shapes of devices 2400a-f in accordance with aspects of the invention. In embodiments, each of the devices 2400a-f comprises an electronic surgical eye implant having smart haptics. The devices 2400a-f may be used as any of the devices 500/600/700 shown in FIGS. 5-7. In embodiments, each of the devices 2400a-f includes a central portion 2410a-f and haptics 2415a-f that is moveably connected to the central portion 2410a-f. In embodiments, the haptics 2415a-f are flexibly connected to the central portion 2410a-f, e.g., by one or more materials that permit flexure between the haptics 2415a-f and the central portion 2410a-f.

Each of the devices 2400a-f may be similar to device 2100 in that it includes a power source, control circuitry, one or more electronic components 2460a-f in the haptics 2415a-f, and an imaging and projection system in the central portion 2410a-f, all of which operate in the same manner as those same elements in device 2100. The imaging and projection system are in the central portion 2410a-f. The power source and control circuitry may be integrated with the imaging and projection system or may be separated from the imaging and projection system. As shown in FIG. 24, the one or more electronic components 2460a-f pass continuously through the haptics 2415a-f and the central portion 2410a-f.

In embodiments, each of the devices 2400a-f is composed of materials that permit it to be arranged in the expanded configuration shown in FIG. 24 and also arranged in collapsed configurations such as those shown in FIGS. 21B and 21C.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A device configured to be implanted in an eye, comprising:
an imaging system that receives visible light incoming to the eye;
a projection system that generates and projects an image onto a retina of the eye in which the device is implanted, the image being based on the received visible light; and
one or more haptics that include one or more electronic components, wherein the one or more haptics are movable relative to a central portion of the device such that the device can be arranged in a collapsed configuration and an expanded configuration.

2. The device of claim 1, wherein the one or more electronic components comprises an inductive charging coil.

3. The device of claim 1, wherein the one or more electronic components comprises a wireless communication antenna.

4. The device of claim 1, wherein the one or more electronic components comprises an inductive charging coil and a wireless communication antenna.

5. The device of claim 1, wherein the one or more haptics are pivotally connected to the central portion.

6. The device of claim 1, wherein the one or more haptics are flexibly connected to the central portion.

7. The device of claim 1, wherein the one or more electronic components extend continuously through the one or more haptics and the central portion.

8. The device of claim 1, wherein the imaging system and the projection system are in the central portion.

9. The device of claim 1, further comprising a power source and control circuitry.

10. The device of claim 9, wherein the power source and/or the control circuitry are integrated with the imaging system and the projection system.

11. The device of claim 9, wherein the power source and/or the control circuitry are separate from the imaging system and the projection system.

12. The device of claim 1, wherein the projection system comprises optical source generation circuitry and an optical phased array that is configured to project the image onto a determined location of the retina.

13. The device of claim 1, wherein the projection system comprises a light generation panel and a microlens array that is configured to project the image onto a determined location of the retina.

14. The device of claim 1, wherein the one or more electronic components comprise flexible electrically conductive elements that maintain their electrical continuity when the device is in the collapsed configuration, when the device is in the expanded configuration, and when the device is moved between the collapsed configuration and the expanded configuration.

15. A method comprising implanting the device of claim 1 into the eye.

16. The method of claim 15, wherein the implanting comprises:
arranging the device in the collapsed configuration;
inserting the device into the eye while the device is in the collapsed configuration; and
arranging the device in the expanded configuration when the device is in the eye.

17. A method of using the device of claim 1, the method comprising:
causing the device to project a diagnostic image on different locations of the retina of the eye;
receiving patient feedback for each of the different locations;
creating a mapping of the retina of the eye based on the feedback; and
programming the mapping into the device.

18. The method of claim 17, further comprising optimizing the mapping using artificial intelligence.

19. The method of claim 17, wherein the mapping maps the retina into functional areas and non-functional areas.

20. The method of claim 19, wherein the device is configured to control the projection system based on the mapping to project a beam onto a functional area of the retina to reduce or eliminate a scotoma caused by a non-functional area of the retina.
